(19) United States
(12) Patent Application Publication
(10) Pub. No.: US 2026/0000847 A1
(43) Pub. Date: Jan. 1, 2026

(54) HEAD MOUNTED SCALP INJECTION DEVICE

(71) Applicant: GOLD NANOTECH INC., Taipei (TW)

(72) Inventor: SHANG-WEN TANG, Taipei (TW)

(73) Assignee: GOLD NANOTECH INC., Taipei (TW)

(21) Appl. No.: 19/256,198

(22) Filed: Jul. 1, 2025

(30) Foreign Application Priority Data
Jul. 1, 2024 (TW) 113124522

(51) Int. Cl. A61M 11/06; A61M 35/00; G16H 40/63; G16H 50/70

(52) U.S. Cl. CPC A61M 11/06; A61M 35/10; G16H 40/63; G16H 50/70; A61M 2205/50; A61M 2209/088; A61M 2210/06

(57) ABSTRACT

The present application provides a head mounted scalp injection device, including: a head mounted device including a headband and at least one pivoting assembly, where the headband is configured to surround and fix on a user's head, and the pivoting assembly is attached to the headband and is pivotable relative to the headband; and an injection device detachably attached to the pivoting assembly so that the injection device is pivotable relative to the user's head, where the injection device is configured to spray a drug toward the user's head by atomizing penetration.

### CROSS-REFERENCE TO RELATED APPLICATION

This non-provisional application claims priority under 35 U.S.C. § 119(a) on Patent Application No(s). 113124522 filed in Taiwan, R.O.C. on Jul. 1, 2024, the entire contents of which are hereby incorporated by reference.

### BACKGROUND OF THE INVENTION

### 1. Field of the Invention

The present disclosure provides a medical device, and in particular, a head mounted scalp injection device.

### 2. Description of the Related Art

Needle-free injection does not use a needle during drug injection. Its principle is to use the high pressure generated by power to make the drug in the syringe penetrate (water column penetration or atomizing penetration) the skin through the nozzle with a jet stream at a high speed of more than 100 meters per second and high pressure to subcutaneous and intradermal tissue layers to release the drug effect.

The conventional needle-free injection device must be operated in a handheld manner, as described in the invention patent No. 1735246 issued in Taiwan. During needle-free injection, the nozzle of the conventional needle-free injection device must be aligned with the position on the scalp where the injection is to be administered. However, the scalp drug administration process needs to last for 10 to 12 minutes. In this process, due to the fatigue of the operator's arm or the random shaking of the injected person's head, the nozzle of the conventional needle-free injection device may deviate from the position where the injection is to be administered, resulting in problems such as the waste of drugs and poor therapeutic effect.

### BRIEF SUMMARY OF THE INVENTION

The inventor has exerted great efforts in careful research, and then developed a head mounted scalp injection device to achieve the objective of long-time fixed-point positioning for administering drugs to the scalp.

The “database element” described throughout the present disclosure refers to a collection of a group of related electronic data and stored in a hard disk, memory or a combination of the above, and can perform relevant processing on the electronic data through the syntax functions provided by the database management system (DBMS), such as adding, reading, searching, updating and deleting, etc. The database management system can manage the electronic data through different data structure methods, such as relational, hierarchical, network or object-oriented, etc. The present disclosure takes the relational database management system as an example for the following description.

The present disclosure provides a head mounted scalp injection device, including: a head mounted device including a headband and at least one pivoting assembly, where the headband is configured to surround and fix on a user's head, and the pivoting assembly is attached to the headband and is pivotable relative to the headband; and at least one injection device detachably attached to the pivoting assembly so that the injection device is pivotable relative to the user's head, where the injection device is configured to spray a drug toward the user's head by atomizing penetration.

In an embodiment, the pivoting assembly includes an arc-shaped bracket and at least one slider, where two opposite ends of the arc-shaped bracket are separately connected to two opposite side surfaces of the headband via a second knob and are pivotable relative to the headband, the second knob is configured to adjust a pivoting angle between the arc-shaped bracket and the headband, the slider is slidably attached to the arc-shaped bracket, and the injection device is fixed to the slider.

In an embodiment, the pivoting assembly includes an arc-shaped support member, where two opposite ends of the arc-shaped support member are separately attached to left and right side surfaces of the headband, and an inclination angle formed between the arc-shaped support member and the headband is less than 90 degrees.

In an embodiment, at least one of the left and right side surfaces of the headband is further provided with a scale, and a second slider is slidable along an extension direction of the scale.

The present disclosure further provides a head mounted scalp injection device, including: a head mounted device including a headband and a cover body, where the headband is configured to surround and fix on a user's head, the cover body is attached to the headband and is configured to cover the user's head, and an inner side of the cover body is provided with at least one slide rail and at least one slider slidably attached to the at least one slide rail; and at least one injection device fixed to the slider so that the injection device is pivotable relative to the user's head, where the injection device is configured to spray a drug toward the user's head by atomizing penetration.

In an embodiment, the headband further includes a first knob for adjusting a size of the headband.

The present disclosure further provides a head mounted scalp injection device, including: a frame including a base and a telescopic rod, where one end of the telescopic rod is attached to the base; a head mounted device including a cover body attached to the telescopic rod and configured to cover a user's head, where an inner side of the cover body is provided with at least one slide rail and at least one slider slidably attached to the at least one slide rail; and at least one injection device fixed to the slider so that the injection device is pivotable relative to the user's head, where the injection device is configured to spray a drug toward the user's head by atomizing penetration.

In an embodiment, the other end of the telescopic rod is provided with a rotating shaft, and the cover body is attached to the rotating shaft so that the cover body is rotatable relative to the telescopic rod.

In an embodiment, a scalp detection device and a processing module are further included, where the scalp detection device is disposed on the inner side of the cover body and is configured to capture and generate a scalp area image of the user's head; the processing module includes a database element, a scalp detection model and an operational element, where the database element is configured to store training sample data including a plurality of scalp area images from each of a plurality of users; the scalp detection model is electrically connected to the database element, where input layer data of the scalp detection model is the training sample data, and output layer data of the scalp detection model is a relative coordinate position between each user's scalp area image and the slider; and the operational element is electrically connected to the scalp detection device, the database element and the scalp detection model, where the operational element is configured to input the scalp area image obtained by the scalp detection device into the scalp detection model to obtain a corresponding relative coordinate position, and the operational element controls the slider to move to the relative coordinate position and then controls the injection device to operate.

In an embodiment, the scalp detection model is implemented by deep learning technology.

Thus, according to the head mounted scalp injection device of the present disclosure, the inconvenience of holding the injection device by hand can be overcome by disposing the injection device on the head mounted device, so as to achieve the effect of long-time fixed-point positioning for administering drugs to the scalp.

### BRIEF DESCRIPTION OF THE DRAWINGS

FIG. 1 is a front view of a first embodiment of the head mounted scalp injection device of the present disclosure.

FIG. 2 is a side view of a first embodiment of the head mounted scalp injection device of the present disclosure.

FIG. 3 is a side view of a first embodiment of the head mounted scalp injection device of the present disclosure with an arc-shaped support member.

FIG. 4 is a usage state diagram showing adjustment of a headband in a first embodiment of the head mounted scalp injection device of the present disclosure.

FIG. 5 is a side sectional view of a second embodiment of the head mounted scalp injection device of the present disclosure.

FIG. 6 is a side sectional view of a head cover in FIG. 5 with a scalp detection device and a processing module added.

FIG. 7 is a block diagram of a system in FIG. 6.

### DETAILED DESCRIPTION OF THE INVENTION

In order to fully understand the objective, features and effects of the present disclosure, the present disclosure will be described in detail by means of the following specific embodiments in conjunction with the accompanying drawings as follows.

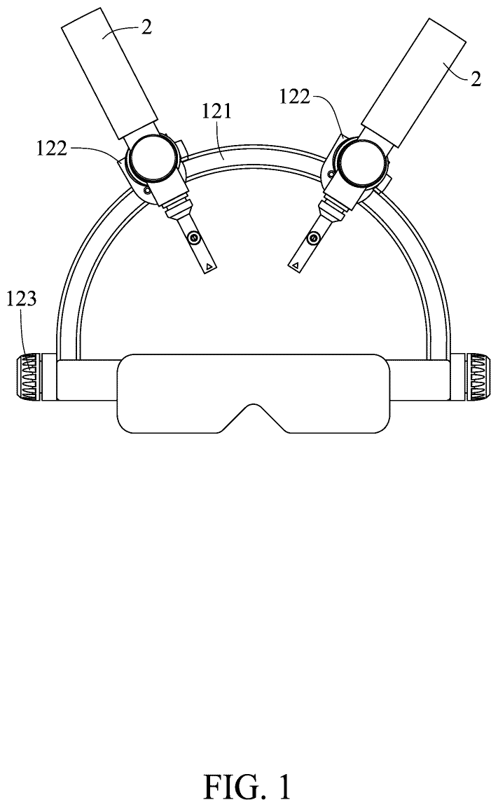

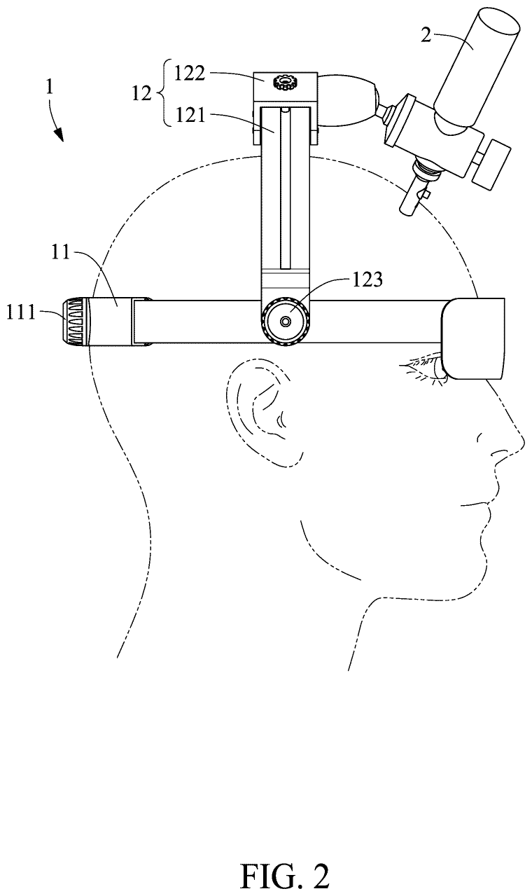

Referring to FIG. 1 and FIG. 2, which are respectively a front view and a side view of a first embodiment of the head mounted scalp injection device of the present disclosure, the head mounted scalp injection device includes: a head mounted device 1 and at least one injection device 2, where the injection device 2 is attached to the head mounted device 1.

In detail, the head mounted device 1 has a headband 11 and at least one pivoting assembly 12, where the headband 11 is configured to surround and fix on a user's head along a first plane, and preferably, the headband 11 may further have a first knob 111 for adjusting a size of the headband 11 to fit users with different head circumferences. In this embodiment, the first knob 111 is disposed at a rear side of the headband 11, and the first knob 111 rotates clockwise/counterclockwise so that a circumference of the headband 11 shrinks/enlarges, but it is not limited thereto.

The pivoting assembly 12 is attached to the headband 11 and is pivotable relative to the headband 11. In this embodiment, the pivoting assembly 12 pivots along a second plane, and the second plane is preferably perpendicular to the first plane.

Specifically, the pivoting assembly 12 has an arc-shaped bracket 121 and at least one slider 122, where two opposite ends of the arc-shaped bracket 121 are separately connected to two opposite side surfaces of the headband 11 and are pivotable relative to the headband 11. In detail, the two opposite ends of the arc-shaped bracket 121 are separately connected to the two opposite side surfaces of the headband 11 by a second knob 123. In this embodiment, the two opposite ends of the arc-shaped bracket 121 are separately connected to left and right side surfaces of the headband 11, and the second knob 123 rotates clockwise/counterclockwise so that the arc-shaped bracket 121 cannot pivot/can pivot relative to the headband 11, but it is not limited thereto. Thus, a pivoting angle between the arc-shaped bracket 121 and the headband 11 can be adjusted by operating the second knob 123.

The slider 122 is slidably attached to the arc-shaped bracket 121. The slider 122 includes an attachment portion (for example: a sleeve hole) to fix the injection device 2. In this embodiment, a combination of the arc-shaped bracket 121 and the slider 122 is an adjustable slide rail fixing element, so as to achieve the function of fixing the slider 122 located on the arc-shaped bracket 121 at any required position on the arc-shaped bracket 121.

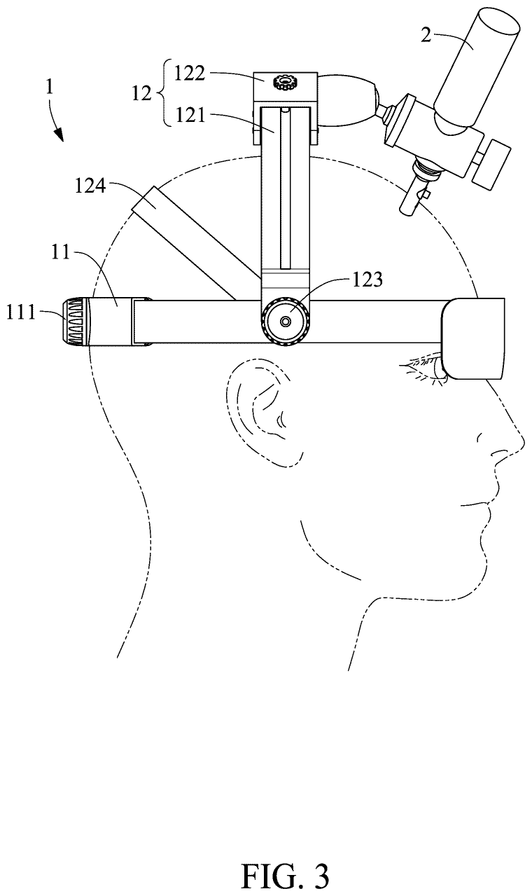

Referring to FIG. 3, the pivoting assembly 12 may further include an arc-shaped support member 124, where two opposite ends of the arc-shaped support member 124 are separately attached to left and right side surfaces of the headband 11. In this embodiment, an inclination angle formed between the arc-shaped support member 124 and the headband 11 is less than 90 degrees. Thus, when a user wears the head mounted device 1, the arc-shaped support member 124 can abut against the back of the user's head, so as to achieve the function of supporting the head mounted device 1.

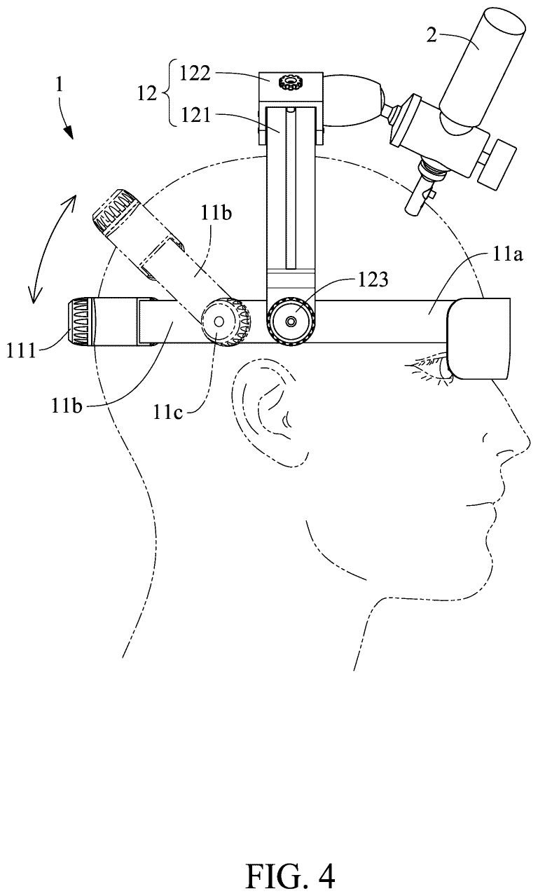

Referring to FIG. 4, the headband 11 may be jointly composed of a first part 11a, a second part 11b and a third knob 11c, where the first part 11a and the second part 11b are separately attached to the third knob 11c, and the third knob 11c rotates clockwise/counterclockwise so that the second part 11b can or cannot pivot relative to the first part 11a, but it is not limited thereto. Thus, a pivoting angle between the second part 11b and the first part 11a can be adjusted by operating the third knob 11c.

The injection device 2 is detachably attached to the attachment portion of the slider 122 of the pivoting assembly 12 so that the injection device 2 is pivotable relative to the user's head. The injection device 2 is configured to spray a drug toward the user's head by atomizing penetration. For example, the injection device 2 can be a needle-free injection device as described in the invention patent No. 1735246 issued in Taiwan.

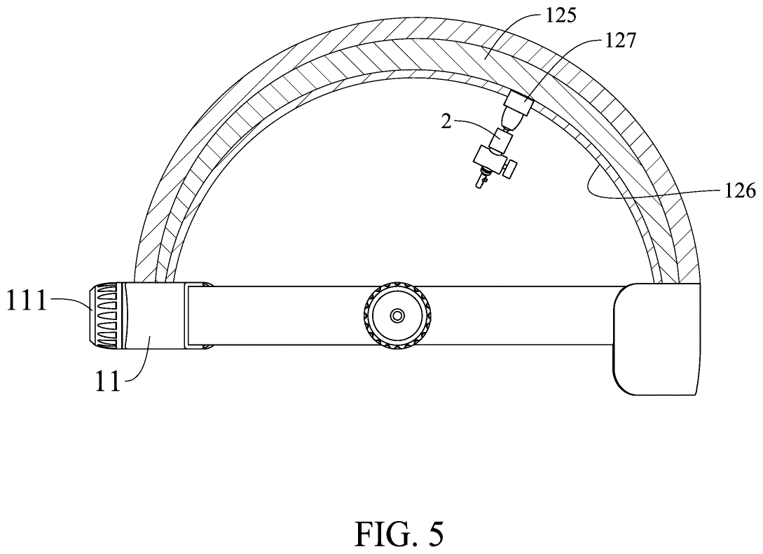

Referring to FIG. 5, which is a side sectional view of a second embodiment of the head mounted scalp injection device of the present disclosure, in this embodiment, the head mounted device 1 includes the above headband 11 and a cover body 125, where the cover body 125 is attached to the headband 11 and is configured to cover a user's head. An inner side of the cover body 125 is provided with at least one slide rail 126 and at least one slider 127 slidably attached to the at least one slide rail 126. The injection device 2 is fixed to the slider 127.

In the second embodiment of the present disclosure, whether relative positions of the injection device 2 and a scalp of the user's head where the drug is to be administered are consistent can be automatically analyzed through artificial intelligence technology. If they are inconsistent, the injection device 2 is controlled to move until the relative positions of the injection device 2 and the scalp of the user's head where the drug is to be administered are consistent.

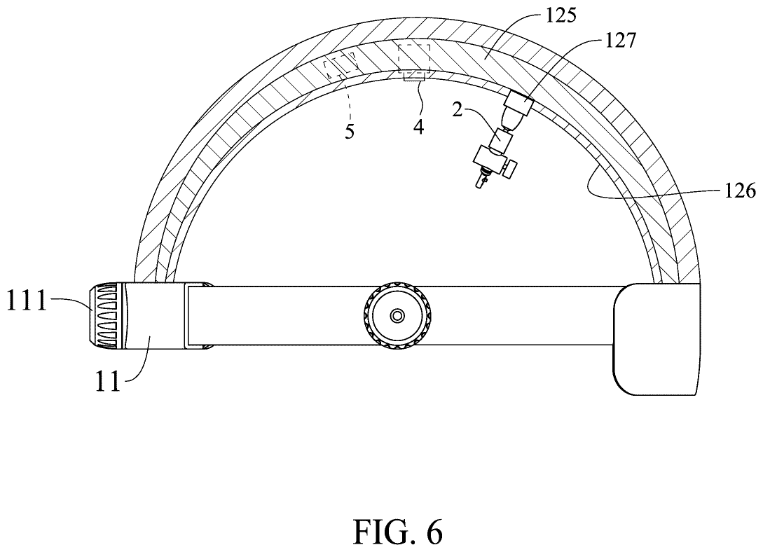

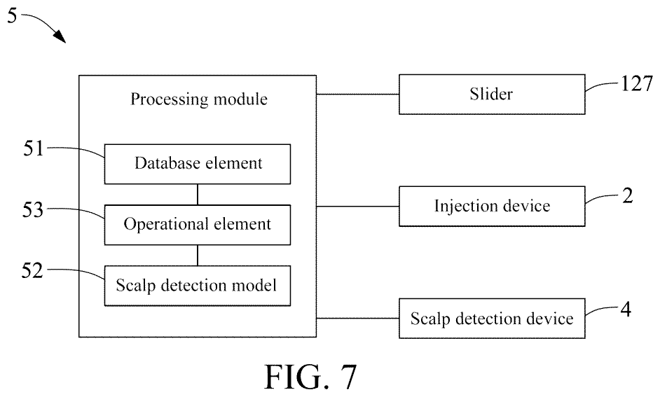

Referring to FIGS. 6 and 7, the head mounted scalp injection device of the present disclosure further includes a scalp detection device 4 and a processing module 5, where the scalp detection device 4 is disposed on the inner side of the cover body 125 and is configured to capture and generate a scalp area image toward a user's head. The scalp detection device 4 can be, for example, a digital microscope. The processing module 5 is disposed on the inner side of the cover body 125 and includes a database element 51, a scalp detection model 52 and an operational element 53, where the database element 51 is configured to store training sample data, including a plurality of scalp area images from each of a plurality of users. It is worth mentioning that in this embodiment, according to the paper “Assessing therapeutic effectiveness of scalp treatments for dandruff and seborrheic dermatitis, part 1: a reliable and relevant method based on the adherent scalp flaking score (ASFS)”, the human scalp can be divided into 8 scalp areas, but it is not limited thereto.

The scalp detection model 52 is electrically connected to the database element 51, where input layer data of the scalp detection model 52 is the training sample data, and output layer data of the scalp detection model 52 is a relative coordinate position between each user's scalp area image and the slider 127. In this embodiment, the scalp detection model 52 is implemented by deep learning technology, and an object detection neural network model such as the YOLO series, RCNN or SegNet can be adopted as a basic architecture. The network architecture and operational process of the object detection neural network model are common knowledge in the art and will not be elaborated here.

In detail, a method for training a training model of the scalp detection model 52 can be supervised learning, and the number of training times of the training model can be set to 1,500 times. The training sample data can be divided into a training dataset and a testing dataset. When the training model is being trained, the training dataset can be further divided into a validation dataset for training and validation. For example, a ratio of the training dataset, the testing dataset and the validation dataset can be 7:2:1, but it is not limited thereto.

The operational element 53 is electrically connected to the scalp detection device 4, the database element 51 and the scalp detection model 52. In this embodiment, the operational element 53 is a microcontroller (MCU). The operational element 53 is configured to input a scalp area image obtained by the scalp detection device 4 into the scalp detection model 52 to obtain a corresponding relative coordinate position. The operational element 53 controls the slider 127 to move to the relative coordinate position and then controls the injection device 2 to operate.

Specifically, a combination of the cover body 125 and the slider 127 is an electric slide rail electrically connected to the operational element 53. The operational element 53 obtains the relative coordinate position and then controls the slider 127 to move to the relative coordinate position. The injection device 2 provides high-pressure gas to an atomizing injection module through a cylinder. The operational element 53 is electrically connected to the cylinder, and after the slider 127 moves to the relative coordinate position, the operational element 53 controls the cylinder to open, so that the injection device 2 sprays out the drug.

Thus, according to the head mounted scalp injection device of the present disclosure, the inconvenience of holding the injection device by hand can be overcome by disposing the injection device on the head mounted device, so as to achieve the effect of long-time fixed-point positioning for administering drugs to the scalp.

The present disclosure has been disclosed with preferred embodiments above. However, those skilled in the art should understand that these embodiments are only used to depict the present disclosure and should not be construed as limiting the scope of the present disclosure. It should be noted that all variations and substitutions equivalent to the embodiments should be included within the scope of the present disclosure. Therefore, the scope of protection of the present disclosure shall be defined by the claims.

What is claimed is:

1. A head mounted scalp injection device, comprising:
a head mounted device comprising a headband and at least one pivoting assembly, wherein the headband is configured to surround and fix on a user's head, and the pivoting assembly is attached to the headband and is pivotable relative to the headband; and
at least one injection device detachably attached to the pivoting assembly so that the injection device is pivotable relative to the user's head, wherein the injection device is configured to spray a drug toward the user's head by atomizing penetration.

2. The head mounted scalp injection device according to claim 1, wherein the headband further comprises a first knob for adjusting a size of the headband.

3. The head mounted scalp injection device according to claim 1, wherein the pivoting assembly comprises an arc-shaped bracket and at least one slider, wherein two opposite ends of the arc-shaped bracket are separately connected to two opposite side surfaces of the headband via a second knob and are pivotable relative to the headband, the second knob is configured to adjust a pivoting angle between the arc-shaped bracket and the headband, the slider is slidably attached to the arc-shaped bracket, and the injection device is fixed to the slider.

4. The head mounted scalp injection device according to claim 1, wherein the pivoting assembly comprises an arc-shaped support member, wherein two opposite ends of the arc-shaped support member are separately attached to left and right side surfaces of the headband, and an inclination angle formed between the arc-shaped support member and the headband is less than 90 degrees.

5. A head mounted scalp injection device, comprising:
a head mounted device comprising a headband and a cover body, wherein the headband is configured to surround and fix on a user's head, the cover body is attached to the headband and is configured to cover the user's head, and an inner side of the cover body is provided with at least one slide rail and at least one slider slidably attached to the at least one slide rail; and
at least one injection device fixed to the slider so that the injection device is pivotable relative to the user's head, wherein the injection device is configured to spray a drug toward the user's head by atomizing penetration.

6. The head mounted scalp injection device according to claim 5, wherein the headband further comprises a first knob for adjusting a size of the headband.

7. The head mounted scalp injection device according to claim 6, further comprising a scalp detection device and a processing module, wherein the scalp detection device is disposed on the inner side of the cover body and is configured to capture and generate a scalp area image of the user's head; the processing module comprises a database element, a scalp detection model and an operational element, wherein the database element is configured to store training sample data comprising a plurality of scalp area images from each of a plurality of users; the scalp detection model is electrically connected to the database element, wherein input layer data of the scalp detection model is the training sample data, and output layer data of the scalp detection model is a relative coordinate position between each user's scalp area image and the slider; and the operational element is electrically connected to the scalp detection device, the database element and the scalp detection model, wherein the operational element is configured to input the scalp area image obtained by the scalp detection device into the scalp detection model to obtain a corresponding relative coordinate position, and the operational element controls the slider to move to the relative coordinate position and then controls the injection device to operate.

8. The head mounted scalp injection device according to claim 7, wherein the scalp detection model is implemented by deep learning technology.

9. The head mounted scalp injection device according to claim 5, further comprising a scalp detection device and a processing module, wherein the scalp detection device is disposed on the inner side of the cover body and is configured to capture and generate a scalp area image of the user's head; the processing module comprises a database element, a scalp detection model and an operational element, wherein the database element is configured to store training sample data comprising a plurality of scalp area images from each of a plurality of users; the scalp detection model is electrically connected to the database element, wherein input layer data of the scalp detection model is the training sample data, and output layer data of the scalp detection model is a relative coordinate position between each user's scalp area image and the slider; and the operational element is electrically connected to the scalp detection device, the database element and the scalp detection model, wherein the operational element is configured to input the scalp area image obtained by the scalp detection device into the scalp detection model to obtain a corresponding relative coordinate position, and the operational element controls the slider to move to the relative coordinate position and then controls the injection device to operate.

10. The head mounted scalp injection device according to claim 9, wherein the scalp detection model is implemented by deep learning technology.
